SEQUENCE LISTING (2 sequences)

SEQ ID NO: 1 - DNA, 33 bp, Artificial Sequence
  oligonucleotide primer
gcgaagcttccctcgggaagatgaatgaatacg

SEQ ID NO: 2 - DNA, 30 bp, Artificial Sequence
  oligonucleotide primer
gcggatatctcaacgtgcggtaagcaccgg

(19) United States
(12) Patent Application Publication
(10) Pub. No.: US 2003/0162691 A1
(43) Pub. Date: Aug. 28, 2003

(54) Novel antimicrobial therapies

(76) Inventor: Arthur Kornberg, Portola Valley, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/386,246

(22) Filed: Mar. 10, 2003

Related U.S. Application Data
(62) Division of application No. 09/896,919, filed on Jun. 28, 2001, now abandoned.
(63) Continuation-in-part of application No. 09/293,673, filed on Apr. 16, 1999, now abandoned.
(60) Provisional application No. 60/082,153, filed on Apr. 17, 1998.

(51) Int. Cl. A61K 31/00; C12N 1/21

(52) U.S. Cl. 514/1; 435/252.3

(57) ABSTRACT

Methods and compositions are provided for treating a host suffering from a disease associated with the presence of a pathogenic microorganism. In the subject methods, a pharmaceutical formulation comprising an agent that at least reduces the amount of polyphosphate in said microorganism is administered to said host. The subject methods and compositions find use in the treatment of a variety of disease conditions.

### CROSS REFERENCE TO RELATED APPLICATIONS

This application is a continuation-in-part of application of application Ser. No. ,09/293,673 filed Apr. 16, 1999; which application claims priority pursuant to 35 U.S.C. § 119 (e) to the filing date of the U.S. Provisional Patent Application Serial No. 60/082,153 filed Apr. 17, 1998, the disclosures of which applications are herein incorporated by reference.

### ACKNOWLEDGMENT

This invention was made with United States Government support under Grant No. GM07581-34 awarded by National Institutes of Health. The United States Government has certain rights in this invention.

### INTRODUCTION

1. Technical Field

The field of this invention is pathogenic microbes and diseases associated therewith.

2. Background of the Invention

Pathogenic microorganisms, e.g. bacteria, are the cause of many different disease conditions. Examples of diseases which result from the presence of pathogenic bacteria include: pneumonia, typhoid, diarrhea, tuberculosis, as well as other bacterial based infections.

To combat such diseases, the pharmaceutical community has developed a number of different antibiotic agents, which agents have revolutionized the practice of medicine. Such agents include: amikacin, gentamicin, tobramycin, amoxicillin, amphotericin B, ampicillin, atovaquone, azithromycin, cefazolin, cefepime, cefotaxime, cefotetan, cefpodoxime, ceftazidime, ceftizoxime, ceftriaxone, cefuroxime, cephalexin, chloramphenicol, clotrimazole, ciprofloxacin, clarithromycin, clindamycin, dicloxacillin, doxycycline, erthromycin lactobionate, imipenem, izoniazid, metronidazole, nafcillin, nitrofurantoin, nystatin, penicillin, pentamidine, piperacillin, rifampin, ticarcillin, trimethoprim, vancomycin, and the like. As such, a large number of antibiotic agents are available for use by the medical practitioner when faced with the treatment of a pathogenic microorganism based disease.

While such agents are effective against pathogenic bacteria and therefor useful in the treatment of disease conditions associated with the presence of such bacteria, there is increasing evidence that certain strains of bacteria are becoming resistant to one or more of the known antibiotic agents. For example, enterococci that are resistant to a vast array of antimicrobial drugs, including cell wall active agents, aminoglycosides, penicillin, ampicillin, and vancomycin, have been observed. Many believe that the emergence of drug resistant bacteria is the result of antibiotic overuse and have thus called for the controlled and limited use of antibiotic agents.

While such an approach to antibiotic use may help slow the problem of microbial drug resistance, new antimicrobial agents must be discovered to combat those strains that are now resistant to most, if not all, currently available antibiotics. As such, there is a continued interest in the identification of novel antimicrobial agents which can be used to further supplement the medical practitioner's armamentarium against pathogenic microorganisms. Ideally, such new agents should have limited, if no, side effects.

Relevant Literature

Articles discussing inorganic polyphosphate include: Crooke et al., “Genetically Altered Levels of Inorganic Polyphosphate in Escherichia coli,” J. Biol. Chem (1994) 269: 6290-6295; Castuma et al., “Inorganic Polyphosphate in the Acquisition of Competence in Escherichia Coli,” J. Biol. Chem (1995) 270: 12980-12983; Rao & Kornberg, “Inorganic Polyphosphate Supports Resistance and Survival of Stationary Phase Escherichia coli,” J. Bacteriol. (1996) 27: 27146-27151; Kuroda et al., “Guanosine Tetra- and Pentaphosphate Promote Accumulation of Inorganic Polyphosphate in Escherichia coli,” J. Biol. Chem. (1997) 272:21240-21243; as well as Kornberg, “Inorganic Polyphosphate: A Molecular Fossil Come to Life,” in Phosphate in Microorganisms: Cellular and Molecular Biology, (Tonriani-Gorini et al., eds, ASM Press, Washington D.C.)(1994) pp 204-208; and Komberg, “Inorganic Polyphosphate: Toward Making a Forgotten Polymer Unforgettable,” J. Bacteriol. (1995) 177: 491-496.

Articles describing polyphosphate kinase include: Ahn & Kornberg, “Polyphosphate Kinase from Escherichia coli,” J. Biol. Chem. (1990) 265: 11734-11739; and Akiyama et al., “The Polyphosphate Kinase Gene of Escherichia coli: Isolation and Sequence of the ppk Gene and Membrane Location of the Protein,” J. Biol. Chem. (1992) 267: 22556-22561. Articles describing exopolyphosphatase include: Akiyama et al., “An Exopolyphosphatase of Escherichia coli: The Enzyme and its ppx Gene in a Polyphosphate Operon,” J. Biol. Chem. (1993) 268: 633-639; and Wurst & Kornberg, “A Soluble Exopolyphosphatase of Saccharomyces cerevisiae; Purification and Characterization,” J. Biol. Chem. (1994) 269: 10996-11001.

Also of interest is: Tinsley & Gotschlich, “Cloning and characterization of the meningococcal polyphosphate kinase gene: production of polyphosphate synthesis mutants,” Infect Immun (May 1995)63(5):1624-30.

### SUMMARY OF THE INVENTION

Methods and compositions are provided for treating a host suffering from a disease associated with the presence of a pathogenic microorganism. In the subject methods, a pharmaceutical composition comprising an agent that at least reduces the amount of inorganic polyphosphate (hereinafter designated polyphosphate) in said microorganism is administered to said host. The subject methods and compositions find use in the treatment of a variety of disease conditions.

### BRIEF DESCRIPTION OF THE DRAWINGS

FIG. 1 depicts the general strategy for the generation of ppk mutants.

FIG. 2 graphs the growth of S.typhimurium FIRN wild type and the ppk mutant derivative in high-Pᵢ (2 mM MOPS) medium.

FIG. 3 graphs the viabilities of the S. typhimurium FIRN wild type and ppk mutant derivative strains in response to heat shock (55° C.).

FIG. 4 graphs the growth of the S.dublin wild type and ppk mutant derivative strains in high-Pᵢ (2 mM) MOPS medium.

FIG. 5 graphs the long term stationary phase survival of the S.dublin wild type and ppk mutant derivative strains in rich (LB) medium.

FIG. 6 graphs the growth of S.flexneri wild type and ppk mutant derivative strains in rich (LB) medium.

FIG. 7 graphs the stationary phase survival of the E.coli MG1655 wild type and ppk ppx mutant strains in rich (LB) medium.

FIGS. 8A to 8E. PPK inhibition by small molecule inhibitors

FIG. 9. Effect of PPK inhibitors on the polyP accumulation

### DESCRIPTION OF THE SPECIFIC EMBODIMENTS

Methods and compositions are provided for treating a host suffering from a disease condition associated with the presence of a microorganism. In the subject methods, a pharmaceutical composition of an active agent that at least reduces the amount of polyphosphate in said microorganism is administered to said host. The subject methods and compositions find use in the treatment of a variety of disease conditions.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

In the subject methods, a microorganism is contacted with an agent in a manner sufficient to at least reduce the amount of polyphosphate in the microorganism, i.e. the intracellular amount of polyphosphate. Microorganisms of interest are typically bacteria, where such bacteria are those that are characterized by the presence of substantial amounts of intracellular polyphosphate and are associated with a disease condition being experienced by the host in which they are present. Generally the bacteria will contain a ppk gene that expresses a PPK enzyme capable of producing polyphosphate from a phosphate precursor. A gene is a PPK gene for purposes of this application if it encodes an enzyme product resembling the amino acid sequence of E.coli polyphosphate kinase, as reported in Akiyama et al., J. Biol. Chem. (1992) 267: 22556, an enzyme having an amino acid sequence that is substantially identical thereto, or an enzyme that is a homolog thereof, where two enzymes are homologs if they share at least about 30% amino acid sequence identity, as determined using the BLAST algorithm, as described in Altschul et al. (1990),J. Mol. Biol. 215:403-10 (using the published default settings, i.e. parameters w=4 and t=17). Representative bacteria include: Campylobacter coli, Helicobacter pylori, Deinococcus radiodurans, Synechocystic-sp, Klebsiella aerogenes, Vibrio cholerae, Escherichia coli, Mycobacterium tuberculosis, Acinetobacter calcoaceticus, Pseudomonas aeruginosa, Neisseria meningitidis, Salmonella typhimurium, Streptomyces coelicolor, Mycobacterium leprae, Shigella dysenteriae and the like.

By contact is meant that the agent and the microorganism are brought within sufficient proximity of one another such that the agent is capable of exerting the desired effect on the intracellular level of polyphosphate in the microorganism. Contact may be achieved in any convenient manner, such as placing the agent in the same environment as the microorganism, and the like.

The agent that is contacted with the microorganism is one that at least reduces the amount of polyphosphate in the microorganism, i.e., the amount of intracellular polyphosphate. By “at least reduces” is meant that contact of the agent with the microorganism results in a decrease of the amount of polyphosphate in the microorganism as compared to a control situation or value. The amount of reduction may vary, but will typically be to a level at, or below, detection (100 picomoles/mg protein). In certain instances, internalization of the agent by the microorganism may result in a decrease of polyphosphate such that substantially no polyphosphate is present in the microorganism, by which is meant that the amount of polyphosphate in the microorganism.

The agent that is contacted with the microorganism is one that acts to decrease the amount of polyphosphate in the microorganism as compared to a control, i.e. the amount of polyphosphate that would be present in the microorganism in the absence of contact with the agent. Generally, the agent will modulate the activity of an enzyme which participates in the life cycle of polyphosphate in the microorganism, e.g. an enzyme responsible for polyphosphate production, e.g. a polyphosphate kinase, or an enzyme response for the degradation of polyphosphate, e.g. an exopolyphosphatase.

Of particular interest in a first embodiment of the subject invention are agents that at least reduce the activity of a polyphosphate kinase, where by reduce is meant that the agents at least diminish the activity of the kinase in the microorganism as compared to a control, where the activity is diminished to near levels of detection (100 units/mg protein) where in certain situations substantially all polyphosphate kinase activity in the microorganism is stopped by administration of the agent. As such, included in this embodiment are agents that substantially inhibit the activity of polyphosphate kinase in the microorganism. The term “agent” as used herein describes any molecule, e.g., protein or pharmaceutical, with the capability of modulating, preferably reducing, the polyphosphate kinase activity of the enzyme. A variety of different agents may be used in this embodiment of the subject invention, where such agents include: inhibitors of the target enzyme that act directly on the target enzyme (e.g., by binding to the target enzyme) such small molecule inhibitors of the enzyme, modulators of the expression of the enzyme, e.g. antisense (which modulate expression by binding to mRNA encoding the target enzyme) and other expression modulators, and the like, as described in greater detail below.

In certain embodiments of interest, the agent is an agent that does not genetically modify the target microorganism. By genetically modify is meant that the agent modifies the genome of the target microorganism, e.g., by disrupting coding sequences of the target enzymes (for example, with a nucleic acid agent that homologously recombines with the genomic DNA of the microorganism), or introduces additional expressed copies of the coding sequence of the target enzyme into the target microorganism, e.g., by introducing non-integrating plasmids containing copies of the coding sequence of for the target enzyme that are expressed in the microorganism. Agents that modulate expression by some mechanism other than disruption of the genomic DNA or providing extra coding sequences, e.g., by interacting with MRNA encoding the target enzyme, e.g., antisense agents, etc., are not agents genetically modify the microorganism.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Such agents can be identified using any convenient screening methodology. In many such assays, a common feature is the contact of polyphosphate kinase with the candidate agent in the presence of polyphosphate precursor, i.e. adenosine triphosphate (ATP), and the amount of polyphosphate that is produced is compared to a control. Typically, the ATP is labeled to provide for ease of detection, where suitable labels include radioisotopic labels, and the like.

Also of interest are modulators of expression of the polyphosphate kinase enzyme, preferably agents which downregulate the expression of this enzyme. Antisense molecules can be used to down-regulate expression of the enzyme. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Of particular interest in a second embodiment of the invention are agents that enhance the activity of exopolyphosphatase in the microorganism, where enhancement of activity is measured by at least an increase the degradation rate of polyphosphate in the microorganism as compared to a control value, where the amount of increase may range from about 500 to 5000 units/mg protein. Agents that find use in this embodiment of the subject invention include agents that increase the expression of the enzyme in the microorganism, agents that introduce additional copies of the gene encoding the enzyme into the microorganism, e.g. plasmids, and the like.

In certain embodiments, a combination of the two different types of agents, i.e. PPK activity inhibitors and PPX activity enhancers, may be employed.

The subject methods find use making a variety of phenotypic changes in the microorganism. Such changes include changes which are beneficial to the host in which the microorganism is present, and include: a reduction in the virulent factor production of the microorganism; a reduction in the enteroinvasiveness of the microorganism; a reduction in motility, e.g., flaggellar motility, a reduction in the microorganism's ability to participate in “attachment and effacement,” a mechanism known to those of skill in the art; a reduction in the growth rate of the pathogen; a reduction in the ability of the organism to produce or form a biofilm, an increase in the susceptibility of the pathogen to pathogen toxic agents, e.g. antibiotic agents; and the like.

As mentioned above, the subject methods find use in treating a host suffering from a disease condition associated with the presence of a microorganism. By treatment is meant that at least the symptoms suffered by the host due to the presence of the microorganism are at least reduced or ameliorated, e.g. their magnitude is at least diminished, as compared to a control, e.g. in an untreated host. Treatment as used herein also includes the substantially complete removal of all symptoms experienced by the host, and includes situations where the host may be said to be cured of the disease condition associated with the presence of the microorganism.

In treating a host suffering from a disease condition according to the present invention, an effective amount of the agent as described above is administered to the host in a manner sufficient such that the requisite contact and internalization of the agent by the microorganism, as described above, occurs. “Effective amount” as used herein means a dosage sufficient to produce a desired result. Generally, the desired result is at least a reduction in the amount of intracellular polyphosphate in the microorganism as compared to a control, where the magnitude of reduction is sufficient to result in treatment of the host. At least an amelioration of the symptoms associated with the pathological condition afflicting the host, where amelioration is used in a broad sense to refer to at least a reduction in the magnitude of a parameter, e.g. symptom, associated with the pathological condition being treated, such as inflammation, congestion, toxin level, and the like, results from administration of the effective amount of active agent. As such, administration of the effective amount of active agent may results in situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In the subject methods, the active agent(s) may be administered to the host using any convenient means capable of resulting in the desired reduction of microorganism intracellular polyphosphate. Thus, the inhibitors can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the compounds can be achieved in various ways, including oral, buccal, rectal, parenteral, intraperitoneal, intradermal, transdermal, intracheal,etc., administration.

The agents employed in the present invention can be administered alone, in combination with each other, or they can be used in combination with other known antimicrobial agents for combination therapy, where examples of such agents are given in the background section above. In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

For nucleic acid compositions, such compositions may be administered by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the nucleic acid composition and then bombarded into skin cells.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The dose of active agent that is administered will necessarily depend on the nature of the active agent, the target microorganism and the host. Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means.

In certain embodiments, the subject ppk/ppx modulatory agents. may be administered in combination with other agents having antimicrobial activity to achieve improved results, e.g. enhanced activity of the antimicrobial agent as compared to administration of the antimicrobial agent by itself, synergistic results, etc. Antimicrobial agents with which the subject compounds may be administered in certain embodiments of the invention include antibiotics, such as: aminoglycosides, e.g. amikacin, apramycin, arbekacin, bambermycins, butirosin, dibekacin, dihydrostreptomycin, fortimicin, gentamicin, isepamicin, kanamycin, micronomcin, neomycin, netilmicin, paromycin, ribostamycin, sisomicin, spectinomycin, streptomycin, tobramycin, trospectomycin; amphenicols, e.g. azidamfenicol, chloramphenicol, florfenicol, and theimaphenicol; ansamycins, e.g. rifamide, rifampin, rifamycin, rifapentine, rifaximin; β-lactams, e.g. carbacephems, carbapenems, cephalosporins, cehpamycins, monobactams, oxaphems, penicillins; lincosamides, e.g. clinamycin, lincomycin; macrolides, e.g. clarithromycin, dirthromycin, erythromycin, etc.; polypeptides, e.g. amphomycin, bacitracin, capreomycin, etc.; tetracyclines, e.g. apicycline, chlortetracycline, clomocycline, etc.; synthetic antibacterial agents, such as 2,4-diaminopyrimidines, nitrofurans, quinolones and analogs thereof, sulfonamides, sulfones; and the like.

A variety of different disease conditions may be treated according to the subject invention. Such disease conditions include: typhoid, gastroenteritis, infantile diarrhea, chronic gastritis, gastric cancer, peptic ulcers, pneumonia, meningitis, dysentery and the like, where common to such disease conditions is the presence of a pathogenic microorganism.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of inhibitor, either in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Also provided are mutant pathogens that at least lack a functional ppk gene, i.e. are at least ppk knockouts, where the subject mutant pathogens may or may not also be a ppx knockout. The mutant pathogens are characterized by having a deletion or insertion mutation in at least their ppx gene, where the subject mutants may further include one more marker genes, e.g. antibiotic resistance genes (such as kan^{r}) positioned inside of the endogenous disrupted ppk gene. The subject mutants are characterized by having a PPK activity that is less than 50%, usually less than 20%, more usually less than 10% and often less than 5% of the PPK activity observed in wild type counterparts, where PPK activity is determined as discussed in the Experimental Section, infra. Specific ppk mutants of the invention include: H.pylori, P.aeruginosa,S.dublin,S.typhimurim,S.flexneri, and V.cholerae. The subject mutants find use in a variety of different applications, including the elucidation of the function of polyP, and the like.

The following examples are offered by way,of illustration and not by way of limitation.

### EXPERIMENTAL

I. Generation of ppk Knockout Mutants

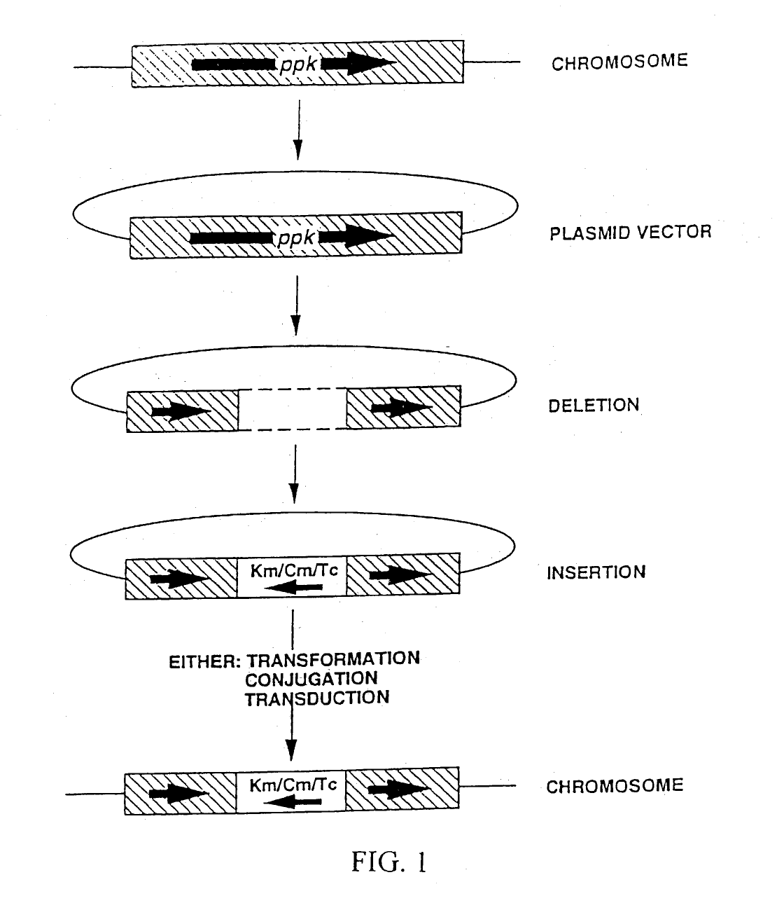

ppk knockout mutants in Helicobacter pylori, Salmonella dublin, Salmonella typhimurium, Shigella flexneri, Vibrio cholerae, Pseudomonas aeruginosa and E.coli were prepared. These mutants were all made by strategies based on either genome databases or on the phylogenetic similarities to E.coli. See FIG. 1. For V. cholerae, H. pylori, and P. aeruginosa, whole or partial genome databases are available for these organisms. The E. coli ppk sequence was used to perform homology searches in each database. Once located, the databases were used to design PCR primers for cloning out the gene. These PCR products were ligated into pBluescript II KS, a common cloning plasmid vector, and major portions (at least ⅔) of each ppk open reading frame was deleted. An antibiotic resistance marker (kanamycin, chloramphenicol, or tetracycline) was then cloned into this site in the opposite orientation of transcription to that of the homologue. These mutations were then cloned into plasmids which cannot replicate in the wild type host. For V. cholerae, H. pylori, and P. aeruginosa, these plasmids were pKNG101, pBluescript II KS, and pBluescript II SK respectively. Wild type strains were then transformed with the appropriate plasmid and transformants were selected and/or screened for double crossover recombination events on solid media. The Salmonella dublin, Salmonella typhimurium and Shigella flexneri mutants were made by taking advantage of their genome similarities to E. coli. For S.flexneri, a P1L4 phage lysate prepared on the Δppk Δppx::kan mutation from E. coli strain CF5802 was used to transduce the mutation into S.flexneri strain 2a. For S. dublin and S.typhimurium, an E. coli Hfr KL16 derivative was constructed which carried the Δppk Δppx::kan mutation from E. coli strain CF5802. This mutation was transferred by conjugation into a Salmonella typhimurium mutL111::Tn10 strain from which a P22 phage lysate was prepared. The mutation was then transduced into S. dublin and transductants were selected on kanamycin plates.

In all of the above cases, a minimum of three mutants were obtained.

The E. coli mutant was prepared in a manner analogous to the protocol described in Crook et al., J Biol Chem 1994 Mar 4;269(9):6290-5 and Rao et al., Bacteriol 1996 Mar; 178(5):1394-400.

Genetic and Biochemical Verification of the Mutants

After screening the desired resistance(s), the mutations were verified by PCR using primers outside the flanking regions and in combination with primers inside the resistance cassettes. Enzyme assays for PPK and PPX activities were performed on all the mutants and parental wild types. The results are presented in Table 1.

| | Specific Activity* | | | | | |
|---|---|---|---|---|---|---|
| | (Units/mg of protein) | | | | Poly P† | |
| | PPK | | | | | |
| | | mu- | PPX | | (nmol/mg) | |
| Organism | w.t. | tant | w.t. | mutant | w.t. | mutant |
| H. pylori | 200 | 15 | 90,000 | 45,000 | 45 | <0.03 |
| P. aeruginosa | 6,400 | 165 | 2,280 | 2,232 | 12 | 0.5 |
| S. dublin | 5,534 | 120 | 475 | <10 | 1.5 | <0.01 |
| S. typhimurim | 5,800 | 12 | N.P. | N.P. | 25 | <1.0 |
| S. flexneri | 3,360 | 86 | 2,911 | <10 | N.P. | N.P. |
| V. cholerae | 17,300 | <300 | N.D. | N.D. | 25.3 | <0.1 |
| E. coli | 3,500 | 150 | 1500 | 150 | 45 | <0.03 |

w.t = wild type
N.P. = not performed
N.D. = not detected
*PPK and PPX activities were measured in rich media. The PPK assays are based on the synthesis of polyP; one unit of PPK is defined as 1 pmol of ³²P ATP phosphate incorporated into polyP per minute at 37° C. PPX assay measured the hydrolysis of polyP; one unit of PPX is defined as 1 pmol of ³²P phosphate liberated from polyP per minute at 37° C.
†PolyP was measured under inducing conditions, i.e. amino acid limitation in minimal medium.

The small residual PPK activities in the mutants indicate that there is an alternate PPK-like activity (PPK2) in these cells. PPK2 has been detected in crude cell extracts of K.pneumoniae and E. coli. PPK2 converts the terminal phosphate of GTP into polyP and is sensitive to phosphate (about 90% inhibition in 1 mM Pᵢ).

III. Phenotypic Assessment of Mutants

A. H.pylori

ppk and ppx do not form an operon in H.pylori. The activity of PPK in the wild type G27 strain is very low compared to the activity observed in E.coli. The G27 ppk mutant strain prepared as described above was tested for urease reactivity, motility, transformability, gastric cell invasion, IL-8 secretion and survival relative to the phenotype. No differences were found between the mutant and the wild type. However, differential interface contrast microscopy revealed significant morphological differences during both mild- and late-exponentional exponential phase and stationary phase. The mutant cells were considerably smaller and had fewer helical turns per cell.

PPK purified from H.pylori G27 was compared with that of E.coli, the specific activity of the H.pylori enzyme is 5-50 times less than that of E.coli. The tetrameric form of PPK is required for the forward reaction, the active enzyme is autophosphorylated and is ammonium sulfate dependent.

The G27 background of the ppk mutant was not suitable for in vivo experiments. Consequently, a new ppk mutant was made in strain SS3 which can be readily tested in the C57 Black mouse line as follows.

Both dose dependent and competition in vivo experiments are performed. In each case, there are two cages with four 6-8 week old C57 Black mice. For the former experiment, the wild type and the mutant H.pylori strains are administered by gavage at ca. 5×10⁸ cfu. Mice are sacrificed at 1 month and their whole stomachs and pylori are examined for pathology. For the latter experiment, equal doses of ca. 2.5×10⁸ cfu of the mutant and wild type are administered similarly and are treated similarly.

B. Salmonella ssp.

In Salmonella species, ppk and ppx presumably form an operon. As such, a deletion/insertion mutation in ppk has a polar effect on the expression of ppx. This is consistent with the results observed as reported in Table 1, above.

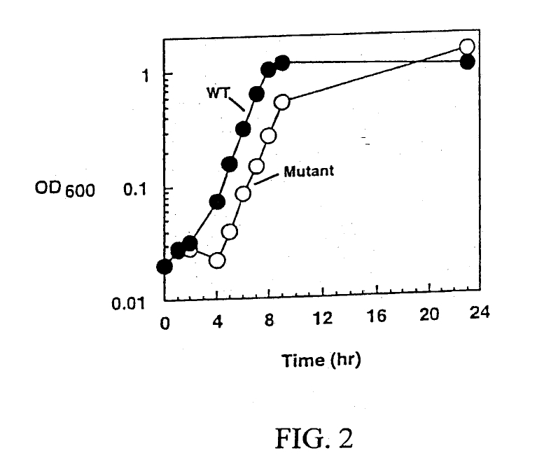

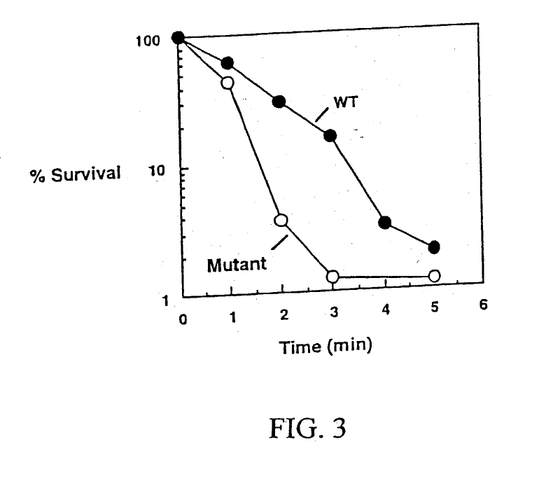

The ppk mutant of S.typhimurium exhibits a growth adaptation defect—about a three-hour lag in growth compared to the wild type, following subculture from LB to either low- or high-phosphate concentration in MOPS was observed. See FIG. 2. No growth differences were observed in the mutant relative to the wild type in rich medium (LB) or in subculture from MOPS to either high- or low-phosphate level MOPS. As in E.coli, the stationary phase culture of the S.typhimurium ppk mutant was significantly sensitive to heat shock (55° C.) relative to the wild type. See FIG. 3. However, no differences were observed between the two strains in long term stationary phase survival or oxidative stress (16 mM hydrogen peroxide).

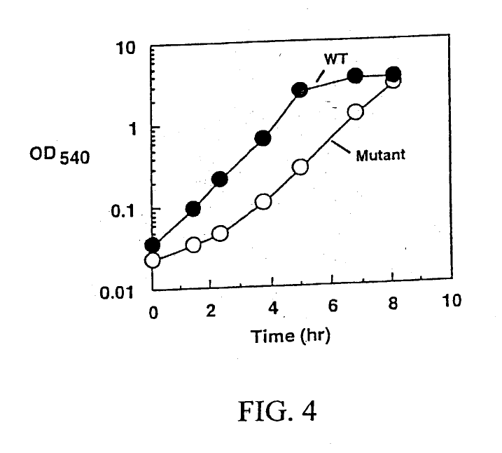

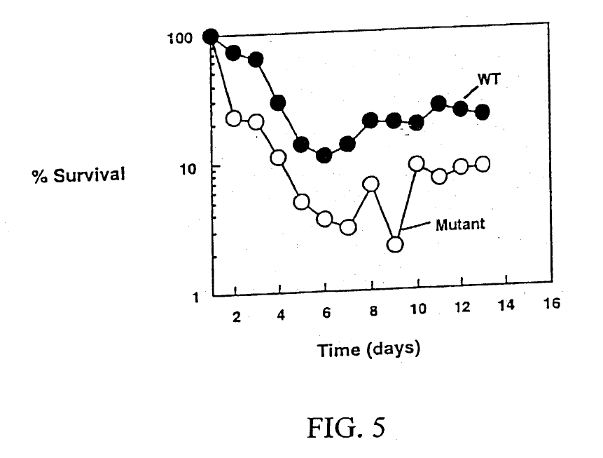

Compared to the isogenic wild type parental strain, the S.dublin mutant shows a growth lag in high-Pi MOPS minimal medium (FIG. 4). The mutant also exhibits a significant reduction in long term stationary phase survival, see FIG. 5, a 4-6 hour lag during growth adaptation from rich medium (LB) to high-phosphate minimal salts medium (MOPS), and a reduced resistance of about 20-40% to the antimicrobial peptide, polymyxin B in stationary phase. In vivo virulence assays have so far been limited to BALB/c mice for S.dublin. No difference in antibiotic resistance resistence or pathogenicity has been observed. However, S.dublin is a bovine serotype (not murine) and no survival competition assays have yet been performed.

C. Shigella flexneri

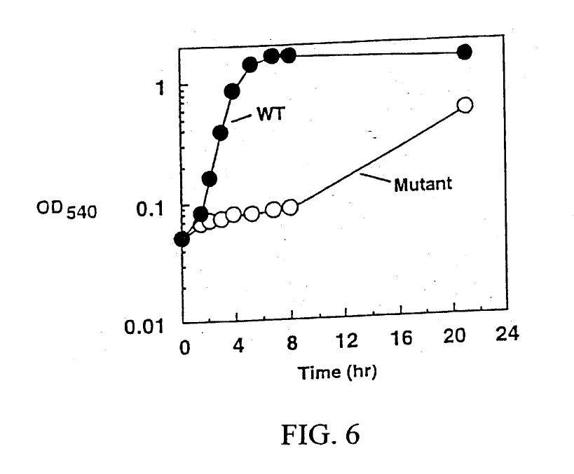

Preliminary studies indicate that ppk and ppx form an operon in S.flexneri such that the ppk deletion/insertion mutation has a polar effect on ppx. As shown in Table 1, the mutant lacks both enzymes. The mutant also exhibits a growth defect in minimal salts (MOPS-buffered) medium and in rich (LB) medium. See FIG. 6.

D. Vibrio cholerae

It was determined that ppk and ppx in V.cholerae form an operon. At the amino acid level, the predicted ppk ORF is 701 residues long (˜81.6 kDa) and is 64% identical to E.coli PPK. Unlike E.coli, V.cholerae accumulates high levels of polyP(>50 nmol (in Pᵢ residues)/mg of total cell protein) when cultivated in a rich (LB) or mineral salts medium. As shown in Table 1, polyP accumulations in the ppk knockout mutant are undetectable. The ppk knockout mutant has been tested for phenotypes (relative to the wild type) under the following conditions: growth rate in MOPS medium with high (2 mM) phosphate and glucose as carbon source, MOPS with sucrose or maltose as carbon sources, MOPS under anaerobic conditions, and in LB. Also tested were growth after shift from Pᵢ-free MOPS to high-Pᵢ MOPS (2 mM) MOPS, growth after shift from LB (exponential phase) to MOPS. Survival was examined in high Pᵢ MOPS, artificial seawater as well as sensitivity to oxidative stress (hydrogen peroxide). Although no phenotype has yet been found for the mutant, it is believed that polyP may be important for the survival of V.cholerae in aquatic and other environments in which it is the source of epidemic outbreaks.

E. P.aeruginosa

In P.aeruginosa, ppk and ppx form two adjacent monocistronic operons convergently transcribed and sharing a 14 bp overlap at their 3′ termini. PPK activity is abolished in the PPK knockout mutants while activity is unchanged in comparison to the wild type. See Table 1. However, polyP accumulates in the mutants to levels at least 10-20% that of the wild type. This residual polyP accumulates in response to low phosphate and amino-acid limitation conditions and upon induction by serine hydroxamate or novobiocin. Persistence of relatively high levels in the mutants is most surprising and requires an examination of factors that control synthesis and removal of residual polyP in this organism.

Phenotypic experiments with the ppk mutant and the wild type revealed no differences under the following conditions: growth in LB or in MOPS with either low (0.1 mM) or high (2 mM) Pᵢ and with or without 2 μg/ml amino acids, growth lag following shift from LB to either low- or high-Pᵢ MOPS, motility on swarm plates, or morphology by phase contrast microscopy.

E. E.coli

The effect of the ppk ppx mutation on biofilm formation in an E.coli MG1655 genetic background was assayed. Biofilm formation was measured both on PVC and polycarbonate surfaces after 18 hr static growth at 30° C. and staining the biofilm with crystal violet as described in Pratt et al., Mol Microbiol (October 1998) 30(2):285-93. Average values derived from 8 independent experiments are provided in Table 2 below.

TABLE 2

| | Growth (OD₅₄₀) | | Biolfim (OD₆₀₀) | |
|---|---|---|---|---|
| | Poly- | Poly- | Poly- | Poly- |
| Strain | vinylchloride | carbonate | vinylchloride | carbonate |
| wild type | 1.87 | 1.03 | 0.34 | 0.42 |
| ppk ppx mutant | 1.44 | 1.16 | 0.27 | 0.18 |

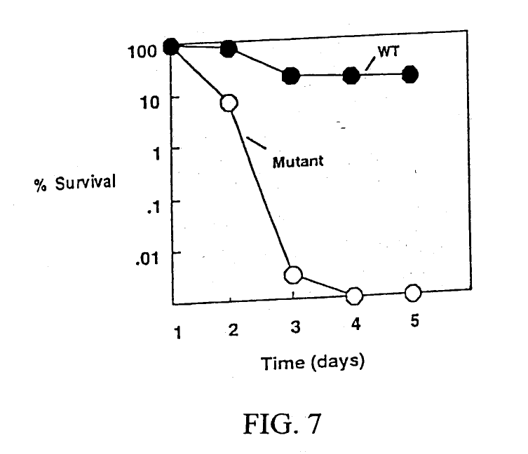

The stationary phase survival of the E.coli MG1655 wild type and ppk ppx mutant strains in rich (LB) medium was also assayed and the results are provided in FIG. 7.

IV. Flagellar Motility Assays

To examine whether the ppk mutation has any effect on the flagellar motility of E.coli, P. aeruginosa, K.pneumoniae, V.cholerae, S.dublin and S.typhimurium, the motility of ppk mutants of these pathogens was compared with the corresponding wild type strains on swim plates containing 0.3% agar. For P aeruginosa, the mutant is severely impaired in motility. This impairment was ascertained by observing swimming motility of P. aeruginosa PAO1 wild type and derivative strains. The flagella-mediated motility of the strains was assessed on tryptone swim plates (1% tryptone, 0.5% NaCl, 0.3% agar) with carbenicillin (300μg/ml) and IPTG (1 mM) after 12 hr of growth at 30° C. Migration of the cells from the point of inoculation (observed asa turbid zone) indicates that a strain is proficient for flagellar-mediated motility. The strains studied were PAO1/p66HE (wild type plus vector control), PAOM-5/p66HE (Δppk plus vector control), PAOM-5/pSCPPX (Δppk plus PPX⁺⁺⁺) and PAOM-5/pPAPPK (Δppk plus PPK⁺⁺⁺). Since the P. aeruginosa ppk mutant still accumulates at least 20% as much polyP under some conditions compared to the wild type, the mutant was transformed with a plasmid overexpressing the yeast exopolyphosphatase (PPX; ScPPX1; Wurst et al., 1995, J Bacteriol 177:898-906) to deplete residual polyP. This strain behaved much like the mutant. When the mutant was transformed with a plasmid expressing P. aeruginosa PPK, the motility was completely restored. This clearly demonstrates the dependence of flagellar motility on PPK function. This observation has been extended to other pathogens (Table 3, supra). Impairments of flagellar motility in the ppk mutants were between 13 to 79% of the wild type levels. As in P. aeruginosa, the motility deficiency of the mutant could be complemented in E. coli by introducing the ppk gene on a plasmid.

Flagellae are highly complex and conserved bacterial organelles requiring coordinated and ordered expression of about 50 genes for their synthesis and function (Shapiro, 1995, Cell 80:525-527). The roles of flagella in chemotaxis and motility are important in the survival of many organisms. A connection between virulence and flagella-based motility has long been observed in many pathogens, some of which require functional flagella for virulence (Moens and Vanderleyden, 1996, Crit Rev Microbiol 22:67- 100; Harshey and Toguchi, 1996, Trends Microbiol 4:226-231; Harshey, 1994, Mol Microbiol 13:389-394) and others in which motility must be suppressed for virulence (Akerley et al., 1995, Cell 80:611-620).

The roles of flagellae and flagella-mediated motility in P. aeruginosa pulmonary and burn infections have been studied in detail (Feldman et al., 1998, Infect Immun 66:43-51; Tang et al., 1996, Infect Immun 64:37-43; Drake et al., 1988, J Gen Microbiol 134:43-52; Montie et al., 1982, Infect Immun 38:1296-1298; Craven and Montie, 1981, Can J Microbiol 27:458-460 ). In the pathogenesis of respiratory tract infection, it has been shown that flagellae and/or flagellar motility is necessary at three distinct stages of infection: (i) acquisition of motile organisms, (ii) immunostimulation, and (iii) adaptation (Feldman et al., 1998, Infect Immun 66:43-51). Flagellar motility and type IV pili-based twitching-motility have been found necessary for the development of a P. aeruginosa biofilm (O'Toole and Kolter, 1998, Mol Microbiol 30:295-304). Bacterial biofilms are troublesome when they form on tissues, on catheters or on medical implants because of their innate resistance to antibiotics and other biocides (Davies et al., 1998, Science 280:295-298). We have found that the ppk mutant of P. aeruginosa is also defective in twitching motility and biofilm formation on abiotic surfaces. As such, PPK or polyP is a key virulence determinant of pathogens, like P. aeruginosa.

In summary, a null mutation in the ppk gene of six bacterial pathogens renders them greatly impaired in motility on semi-solid agar plates; this defect can be corrected by the introduction of ppk gene in trans. In view of the fact that the motility of pathogens is essential to invade and establish systemic infections in host cells (Ottemann and Miller, 1997, Mol Microbiol 24:1109-1117), this impairment in motility is evidence that PPK or polyP plays a crucial and essential role in bacterial pathogenesis.

TABLE 3
Flagella-mediated motility of pathogens on swim plates

| | | Swim area |
|---|---|---|
| Strain | Relevant genotype | (% WT ± SEM)* |
| E. coli MG1655 | WT | 100 ± 7.0 |
| | Δppk − ppx | 46 ± 3.5 |
| | WT + vector | 100 ± 7.3 |
| | Δppk − ppx + vector | 33 ± 4.7 |
| | Δppk − ppx + ppk⁺⁺⁺ | 91 ± 6.6 |
| P. aeruginosa PAO I | WT | 100 ± 12.7 |
| | Δppk | 31 ± 1.8 |
| | WT + vector | 100 ± 8.9 |
| | Δppk + vector | 13 ± 1.7 |
| | Δppk + ppx⁺⁺⁺ | 13 ± 1.2 |
| | Δppk + ppk⁺⁺⁺ | 92 ± 14.7 |
| K pneumoniae ATCC9621 | WT | 100 ± 5.0 |
| | Δppk − ppx | 33 ± 0.7 |
| V. cholerae 92A1552 | WT | 100 ± 4.5 |
| | Δppk | 57 ± 4.8 |
| S. dublin SVA47 | WT | 100 ± 3.6 |
| | Δppk − ppx | 58 ± 3.8 |
| S. typhimurium FIRN | WT | 100 ± 6.4 |
| | Δppk − ppx | 79 ± 8.6 |

*Swim area was measured after 10-12 hr of incubation at 37° C. on tryptone swim plates.

Standard Error of the Mean (SEM) equals σₙ₋₁/\sqrt{n}(Rashid et al, 1995, Microbiology 141: 2391-2404), where n=10, except for P. aeruginosa where incubation was at 30° C. and n=4.

V. Small Molecule Inhibitors of PPK Activity

A. Enzyme purification and assay development:

E.coli PPK was chosen for screening ICOS' chemical library since it is the most well characterized enzyme. The enzyme was purified and the enzyme assay was done according to Ahn, K., and Kornberg A. (1990) Journal of Biological Chemistry, 265, 11734-11739. E.coli lysates overexpressing PPK were fractionated on SP-Sepharose chromatography, followed by Heparin Sepharose. The purified fraction contained a tetramer of 69-kDa PPK polypeptides that reacted with anti-PPK antibodies on immunoblot. To identify a small molecule that inhibits polyP accumulation in bacterial cells, the forward reaction of PPK was focused upon. The reaction was carried out at room temperature for 15 min, and radiolabeled polyP bound to DE-81 membrane was quantitated.

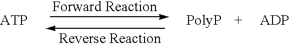

B. Biochemcial characterization of PPK inhibitors:

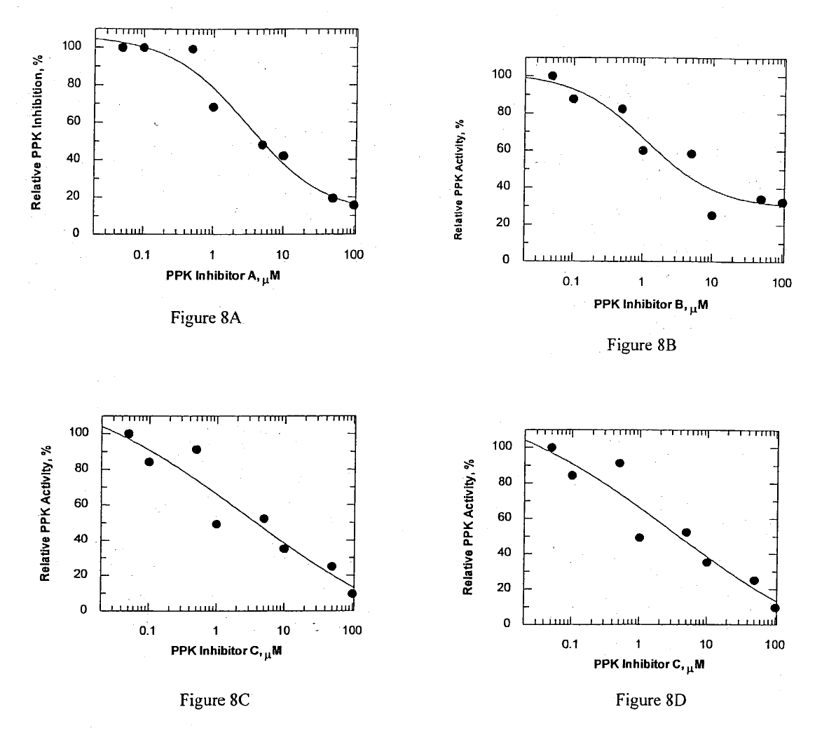

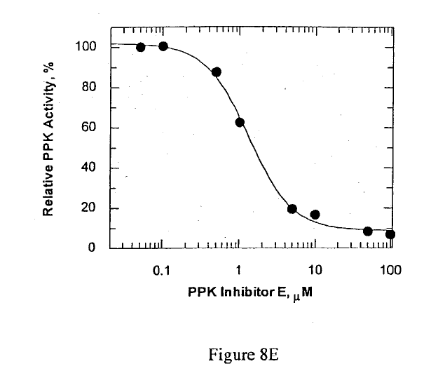

PPK inhibitors identified from ICOS' chemical library were further assayed on analytical thin layer chromatography (PEI-TLC) to verify IC₅₀ values. Table 4 and FIGS. 8A to 8E (FIG. 8A is Inhibitor A; FIG. 8B is Inhibitor B; FIG. 8C is Inhibitor C; FIG. 8D is Inhibitor D; and FIG. 8E is Inhibitor E) show a summary of PPK inhibitors.

TABLE 4
IC₅₀ values of PPK inhibitors

| PPK inhibitors | IC₅₀, μM |
|---|---|
| A | 3.1 |
| B | 1.1 |
| C | 3.29 |
| D | 0.012 |
| E | 1.32 |

C. Inhibition of polyP accumulation in E.coli:

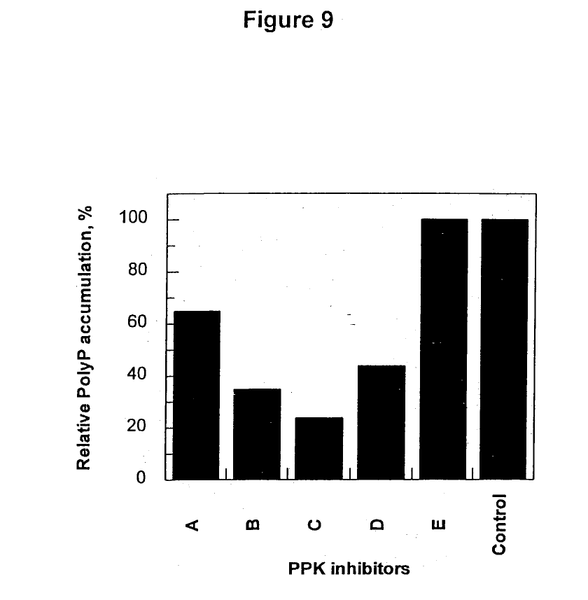

Effects of the PPK inhibitors on the in vivo polyP accumulation were also determined. In order to detect polyP accumulation, cells grown in LB medium were transferred to Mops minimal medium containing low phosphate (10 mM) and 100 μM of each PPK inhibitor. The cells were further incubated for 2 hour, and polyP was subsequently extracted. Relative PolyP accumulation was determined by PPK reverse reaction in the presence of ADP. The results are shown graphically in FIG. 9.

VI. Additional Characterization in Pseudomonas aeruginosa

In this example, we demonstrate that they are essential for quorum sensing and virulence of this clinically important pathogen. This result identifies PPK as a target for the development of a new class of antibacterial drugs.

A. Materials and Methods

1. Bacterial Strains and Plasmids.

P. aeruginosa strains used in this study were PAO1 (wild type; WT) and PAOM5 [ppk::tetracycline resistance (Tc^{R})] (Rashid, M. H., Rao, N. N. & Kornberg, A. (2000)J. Bacteriol. 182, 225-227; Rashid, M. H. & Kornberg, A. (2000) Proc. Natl. Acad. Sci. USA 97, 4885-4890). Plasmid pHEPAK11 for complementation in the PAOM5 strain was constructed as follows. The P. aeruginosa ppk gene was amplified from the plasmid pPPK02F (Ishige, K., Kameda, A., Noguchi, T. & Shiba, T. (1998) DNA Res. 5, 157-162) by PCR with the forward primer 5′-gcgAAGCTTCCCTCGGGAAGATGAATGAATACG-3′(SEQ ID NO:01) (gcg clamp in lowercase and HindIII site in bold, followed by a 24-mer stretch of DNA starting at 154 bp upstream of the GTG translational start codon of the ppk gene (Zago, A., Chugani, S. & Chakrabarty, A. M. (1999) Appl. Environ. Microbiol. 65, 2065-2071) and the reverse primer 5′-gcgGATATCTCAACGTGCGGTAAGCACCGG-3′(SEQ ID NO:02)(gcg clamp in lowercase, EcoRV site in bold and followed by a 21-mer stretch of DNA starting at the TGA stop codon of the ppk gene). The 2.23-kb PCR product was cloned into the HindIII/SmaI site of the E. coli-P. aeruginosa shuttle vector pMMB66HE (Furste, J. P., Pansegran, W., Frank, R., Blocker, H., Scholz, P., Bagdasarian, M. & Lanka, E. (1986) Gene 48, 119-13 1), which places the ppk gene under the control of isopropyl β-D-thiogalactoside-inducible tac promoter. After checking in E. coli DH5α for the overexpression of PPK activity, this plasmid was electroporated into the P. aeruginosa ppk mutant. Resistance to carbenicillin is conferred by pHEPAK11 both in E. coli and in P. aeruginosa.

2. Biofilm Assays.

Static biofilms. Cells were grown in M63 minimal media supplemented with 0.2% glucose, 1 mM MgSO₄, and 0.5% casamino acids (O'Toole, G. A. & Kolter, R. (1998) Mol. Microbiol. 28, 449-461) at 30° C. for static biofilm experiments. (i) Quantitation of biofilm bacteria. Experiments were performed as described earlier (Id.). The strains were cultured in separate wells of a polystyrene microtiter dish followed by staining with crystal violet; the cell-attached dye was solubilized with ethanol and measured at OD₅₉₅. (ii) Epifluorescence and scanning confocal laser microscopy (SCLM). The strains harboring the green fluorescent protein (GFP) expression vector pMRP9-1 (Davies, D. G., Parsek, M. R., Pearson, J. P., Iglewski, B. H., Costerton, J. W. & Greenberg, E. P. (1998) Science 280, 295-298) were grown for 18 h in glass chambers containing borosilicate coverglass (no. 1) bottoms (chambered coverglass systems, Nalge Nunc). The chambers were emptied, washed with water, and examined with a scanning confocal laser microscope (MultiProbe 2010, Molecular Dynamics) using 488- and 510-nm excitation and emission wave lengths, respectively.

Continuous flow-cell biofilm. Flow-cell biofilm experiments were performed in EPRI medium (0.005% sodium lactate/0.005% sodium succinate/0.005% ammonium nitrate/0.00019% KH₂PO₄/0.00063% K₂HPO₄, pH 7.0/0.001% Hunter salts/0.001% L-histidine) (Id.) supplemented with 0.1% dextrose (pH 7.0) in a bioreactor of the once-through, continuous-culture type (Id.). Cell clusters are defined as assemblages of bacteria greater than 10 μm in thickness. Cell-cluster thickness determinations were made by using transmitted light microscopy to determine the base of the biofilm cell cluster at the substratum and the apex of the biofilm cell cluster at the bulk-liquid interface farthest from the substratum. Cell cluster surface area coverage measured the total area in a microscope field occupied by cell clusters.

3. Extraction and Bioassays of AI-1 and AI-2.

Extraction. P. aeruginosa cultures were grown to early stationary phase (OD₆₀₀ of 1.5) in peptone trypticase soy broth at 37° C. with shaking. AI-1 and AI-2 were extracted from the culture supernatants of the ppk mutant with ethyl acetate (Pearson, J. P., Gray, K. M., Passador, L., Tucker, K. D., Eberhard, A., Iglewski, B. H. & Greenberg, E. P. (1994) Proc. Natl. Acad. Sci. USA 91, 197-201).

4. Bioassays.

Bioassays were performed in E. coli reporter strains grown in modified A medium. I-1 bioassays were performed with E. coli MG4 λAI₁4 harboring a lasI:. lacZ fusion in a monolysogen (Seed, P. C., Passador, L. & Iglewski, B. H. (1995) J. Bacteriol. 177, 654-659). AI-2 bioassays were performed in E. coli DH5α harboring the pECP61.5 plasmid containing an rhlA-lacZ fusion construct (Pearson, J. P., Pesci, E. C. & Iglewski, B. H. (1997) J. Bacteriol. 179, 5756-5767). For AI-1 bioassay, a 1-ml overnight culture diluted 1:100 was mixed with the sample and grown for 3-4 h at 37° C. For AI-2 bioassay, overnight cultures were diluted 1:100 and grown at 37° C. with shaking to an OD₆₀₀ of 0.3; 1-ml samples were then further grown 90 min with 1 mM isopropyl β-D-thiogalactoside in the presence of AI-2. Comparison of the β-galactosidase values obtained with the extracted AIs against those of standard curves plotted with the synthetic AIs allowed the estimation of AI content in each sample.

5. Elastase and Rhamnolipid Assays.

For elastase activity measurements, cells were grown in peptone trypticase soy broth at 37° C. for 20 h with shaking; elastase activity was measured by the elastin Congo red assay (Id.). For the measurement of rhamnolipid, cells were grown in modified Guerra-Santos (GS) medium at 37° C. for 80 h with shaking; rhamnose content was determined by oricinol assays and compared to rhamnose standards (Id.).

6. β-Galactosidase Measurements.

Activity was measured as described (Miller, J. H. (1972) Experiments in Molecular Genetics (Cold Spring Harbor Lab. Press, Plainview, N.Y.).). E. coli was grown in modified A medium with shaking at 37° C. to an OD₆₀₀ of 0.5-0.8. P. aeruginsoa was grown in Luria-Bertani medium at 30° C. with shaking for 20 h. Strains PAO1 and PAOM5 harbored either plasmid pTS400 (lasB-lacZ) or pECP60 (rhlA-lacZ) (Pearson, J. P., Pesci, E. C. & Iglewski, B. H. (1997) J.Bacteriol. 179, 5756-5767).

7. Virulence Assays.

Inoculum preparation. P. aeruginosa inocula for virulence studies were prepared as described earlier (Rumbaugh, K. P., Griswold, J. A., Iglewski, B. H. & Hamood, A. N. (1999) Infect. Immun. 67, 5854-5862; Rumbaugh, K. P., Griswold, J. A. & Hamood, A. N. (1999) J.Burn Care Rehab. 20, 42-49). Overnight cultures were diluted 1:100 into fresh Luria-Bertani medium and incubated at 37° C. for 4 h (OD₅₄₀=0.9-1.0.). A sample (100 μl) from each culture was pelleted, washed in PBS, and serially diluted. An aliquot (100 μl) of a 10⁻⁵ dilution was injected containing approximately 200-300 colony-forming units (CFUs) of P. aeruginosa that produces 94-100% lethality by 48 h postburn (Id.).

8. Burned Mouse Model.

Virulence experiments were conducted with adult female ND4 Swiss Webster mice weighing 20-24 g with burn lesions as described earlier (Id.). The mice were anesthetized by i.p. injection of 0.4 ml of 5 mg/ml Nembutal (5% sodium pentobarbital; Abbott). Fluid replacement therapy consisting of a s.c. injection of 0.8 ml 0.9% NaCl solution was administered immediately after the burn. Mice were inoculated s.c. with ≈200 CFU directly under the burn whereas control mice received 100 μl of sterile PBS solution. During recovery, the mice were observed under warming lights. Animals were treated humanely and in accordance with the protocol approved by the Animal Care and Use Committee at Texas Tech University Health Sciences Center in Lubbock, Tex. Three groups of five mice each were burned and infected for each experiment. For in vivo lethality assay, mortality was recorded at 48-h postburn infection. For horizontal spread, mice were killed and two sections (≈5×5 mm each) of the burned skin were removed from each animal at 8- and 24-h postburn infection. One section was obtained from the inoculation site, whereas the other was obtained ≈1.5 cm distant. For systemic spread, livers and spleens of infected mice were collected at 24-h postburn infection.

9. CFU Determinations.

Mouse tissues were suspended in PBS and homogenized, and an aliquot of the homogenate was plated on Luria-Bertani agar plates to determine CFU/g of tissue (Id.).

B. Results and Discussion

1. PPK is Essential for Biofilm Development.

In P. aeruginosa biofilm development, flagella-mediated swimming motility is needed for the initial attachment of individual cells to an abiotic surface followed by microcolony formation mediated by the type IV pili-mediated twitching motility. The subsequent development of elaborate three-dimensional structures requires the quorum-sensing lasR-lasI system. Inasmuch as the ppk mutant is defective in swimming, swarming, and twitching, we measured the capacity of the mutant to attach to and form biofilms on an abiotic surface in a simple, static-culture system that is less dependent on attachment. As expected, the ppk mutant is moderately defective in attachment (at 8 h) to a polystyrene surface. But even past attachment (at 20 h), another defect (presumably maturation of biofilm) was apparent, which was restored to WT level by complementation with the ppk gene. These defects do not arise from growth impairment because the mutant had a growth rate indistinguishable from the WT. Thus, biofilm maturation as well as surface attachment is greatly affected in the ppk mutant.

To compare the architecture of WT and ppk mutant biofilms, a plasmid with a gene encoding an enhanced GFP enabled the viewing of static-culture biofilms by epifluorescence and SCLM. The side view of the 18-h static-biofilm acquired by SCLM revealed adherent clusters of WT cells; they appeared to be in loose aggregates with considerable intervening spaces between them. By contrast with the WT biofilm (105±5 μm), the mutant biofilm was thin (25±1 μm) and much more uniform. A top view generated by epifluorescence microscopy showed the WT cells in clusters as compared to a much more uniform distribution of the ppk mutant. The overall architecture of the 18-h static biofilm is very similar to that of a 2-wk flow-cell biofilm.

In a continuous-flow cell, the three-dimensional architecture of WT biofilms and that of the ppk mutant reached steady-state levels within 10 days, as expected. As with the mature static biofilms, the ppk mutant biofilm was only ≈20% of the WT thickness. The substratum surface area coverage by cell clusters of the mutant biofilm was only 10% (or less) of the WT. Microcolonies composed of groups of WT cells were separated by water channels, whereas the ppk mutant appeared to grow as a continuous sheet on the glass surface. Thus, for the differentiation of mature biofilms, viewed either in static or continuous-flow systems, the ppk mutant is profoundly deficient. This biofilm maturation defect of the ppk mutant is similar to the one that was seen in a lasI mutant deficient in the synthesis of AI-1 quorum-sensing molecule.

2. Effect of the ppk Mutation on the Synthesis of Autoinducers and Extracellular Virulence Factors.

Inasmuch as the ppk mutant is defective in three types of motility, surface attachment and biofilm differentiation, we determined the levels of quorum-sensing molecules AI-1 and AI-2 in the culture supernatant of the ppk mutant by bioassays using E. coli reporter strains. AI-1 and AI-2 levels in the ppk mutant were reduced to ≈50% those of the WT; complementation of the mutant with the ppk gene doubled the WT levels. Clearly, PPK modulates the synthesis of both AIs in P. aeruginosa.

Because the production of extracellular virulence factors including elastase are under quorum-sensing control, we examined qualitatively the level of elastase activity on elastin agar plate as a guide to virulence factor production in the mutant. The activity was reduced in the mutant that could be complemented with the plasmid expressing the ppk gene. The total elastase activity and the total rhamnolipid amount, determined quantitatively in the culture supernatant of the ppk mutant were reduced to 7% and 38% of the WT levels, respectively; complementation with ppk restored the WT levels. With respect to the quorum-sensing target gene lasB for the major elastase and rhlA for a rhamnosyltransferase required for rhamnolipid biosynthesis, their expression in the ppk mutant determined with lacZ fusion constructs, was reduced to 7% for lasB-lacZ whereas that of the rhlA-lacZ was reduced to 3% of the WT levels. These data suggest that PPK and/or poly P affects the quorum-sensing system in the synthesis of AIs and probably also in the formation of AI complexes with cognate regulatory proteins. Alternatively, the ppk mutation, at another level, may affect the transcriptional activation of downstream target genes in interactions with RNA polymerase and σfactors. The drastic effects of the ppk mutation on lasB and rhlA expression might thus represent actions at more than one level

3. PPK Is Necessary for Virulence in the Burned-Mouse Model.

The burned-mouse model was used to examine the effect of the ppk mutation on the pathogenesis of P. aeruginosa infections in burn wounds. The nonlethal burn injury is created by a thermal shock (with 90° C. water for 10 s) to the shaved back of a mouse (≈15% of the body surface area). P. aeruginosa injected s.c. into the center of the burn wound proliferates and invades the underlying tissues. The infection spreads horizontally and systemically. In three separate experiments, only one mouse out of 19 survived inoculation with WT bacteria as compared to the survival of 14 out of 15 inoculated with the ppk mutant. This result for the ppk mutant is the same as observed in the quorum-sensing mutant that lacks both AIs. Complementation of the ppk mutant bacteria with PPK raised the mortality of mice from 7% to 53%. This partial complementation may be due to gene dosage effects because overexpression of PPK in E. coli has been found to be lethal (N. N. Rao & A.K., unpublished result).

With regard to horizontal spread within the burned skin, the ppk mutant bacteria were completely absent at a distant site at 8 h, and a few were found at the inoculation site compared to the WT bacteria. The levels of the mutant bacteria both at inoculation and distant sites at 24 h increased but remained low compared to the WT bacteria. Systemic spread of the ppk mutant to the liver and spleen at 24 h postinfection was <1% that of the WT. The effects of the ppk mutation on both horizontal and systemic spreads are even more drastic than those observed with the quorum-sensing mutant deficient in both AI-1 and AI-2 production.

4. Conclusion.

These studies demonstrate that PPK is essential in P. aeruginosa not only for various forms of motility but also for the development of biofilms, production of the virulence factors elastase and rhamnolipid, and for virulence in the burned-mouse pathogenesis model. All of these effects are likely exerted through a defect in quorum sensing and responses. Because formation of biofilms and their inherent resistance to antimicrobial agents are at the root of many persistent and chronic infections such as the lungs of cystic fibrosis patients, PPK qualifies as a therapeutic target to control biofilm infections by perturbing the integrity of the quorum sensing system. An antimicrobial drug targeted to PPK will enjoy a broad spectrum of activity and little toxicity, inasmuch as the enzyme has not been found in mammalian cells. As PPK is involved in cellular metabolism rather than in an essential function, drugs targeted to it will be less likely to provoke resistance. Because PPK is highly conserved in both Gram-positive and Gram-negative pathogens, the inhibitors of PPK may block quorum-sensing at an upstream level, as opposed to the analogues of specific quorum-signaling molecules in switching off virulence gene expression and thereby attenuating pathogenicity.

It is evident from the above results and discussion that the subject invention provides a novel and much needed antimicrobial therapy. The subject methods and compositions provide an important addition to the armamentarium of weapons available to the doctor for use in the fight against pathogenic microorganisms. Furthermore, since the agents target polyphosphate kinase and exopolyphosphatase enzymes, which enzymes have not been found in mammals, the agents have little or no adverse side effects.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for at least reducing the amount of polyphosphate in a microorganism, said method comprising:
contacting said microorganism with an agent that modulates the activity in said microorganism of an enzyme selected from the group consisting of a polyphosphate kinase and an exopolyphosphatase, wherein said agent does not genetically modify said microorganism.

2. The method according to claim 1, wherein substantially no polyphosphate is present in said microorganism following said contacting step.

3. The method according to claim 2, wherein said agent reduces polyphosphate kinase activity in said microorganism.

4. The method according to claim 2, wherein said agent enhances exopolyphosphatase activity in said microorganism.

5. A method for reducing the expression of virulent factors in a microorganism, said method comprising:
contacting said microorganism with an agent that at least reduces the amount of. polyphosphate in said microorganism as compared to a control, wherein said agent does not genetically modify said microorganism.

6. The method according to claim 5, wherein said agent modulates the activity in said microorganism of an enzyme selected from the group consisting of a polyphosphate kinase and an exopolyphosphatase.

7. The method according to claim 6, wherein said agent reduces the polyphosphate kinase activity in said microorganism.

8. The method according to claim 6, wherein said agent enhances the exopolyphosphatase activity in said microorganism.

9. A method for reducing the virulence of a pathogenic microorganism, said method comprising:
contacting said microorganism with an agent that at least reduces the amount of polyphosphate in said microorganism as compared to a control, wherein said agent does not genetically modify said microorganism.

10. The method according to claim 9, wherein said agent modulates the activity in said microorganism of an enzyme selected from the group consisting of a polyphosphate kinase and an exopolyphosphatase.

11. The method according to claim 9, wherein said agent reduces the polyphosphate kinase activity in said microorganism.

12. The method according to claim 9, wherein said agent enhances the exopolyphosphatase activity in said microorganism.

13. A method of treating a host suffering from a disease associated with the presence of a pathogenic microorganism, said method comprising:
administering to said host an agent that at least reduces the amount of polyphosphate in said pathogenic microorganism as compared to a control wherein said agent does not genetically modify said microorganism.

14. The method according to claim 13, wherein said agent modulates the activity in said pathogenic microorganism of an enzyme selected from the group consisting of a polyphosphate kinase and an exopolyphosphatase.

15. The method according to claim 13, wherein said agent reduces the polyphosphate kinase activity in said pathogenic microorganism.

16. The method according to claim 13, wherein said agent enhances the exopolyphosphatase activity in said pathogenic microorganism.

17. A pharmaceutical composition comprising an agent that at least reduces the amount of polyphosphate in microorganism as compared to a control, wherein said agent does not genetically modify said microorganism.

18. The composition according to claim 17, wherein said agent modulates the activity in said pathogenic microorganism of an enzyme selected from the group consisting of a polyphosphate kinase and an exopolyphosphatase.

19. The composition according to claim 18, wherein said agent reduces the polyphosphate kinase activity in said microorganism.

20. The composition according to claim 18, wherein said agent enhances the exopolyphosphatase activity in said microorganism.

21. A ppk mutant of a pathogen selected from the group consisting of: H.pylori, P.aeruginosa, S.dublin, S.typhimurim, S.flexneri, and V.cholerae.
